(19) United States
(12) Patent Application Publication
(10) Pub. No.: US 2006/0161225 A1
(43) Pub. Date: Jul. 20, 2006

(54) Medical implant system

(75) Inventors: Gerard Wolfe Sormann, North Caulfield (AU); Simon Michael West, Williamstown (AU); Nicholas Victor Zohan Shuley, East Melbourne (AU); Dinesh Kant Kumar, Keilor East (AU); Rodney Bruce Waterhouse, North Fitzroy (AU); Alan Bernard Bradley, Lara (AU)

Correspondence Address: MICHAEL BEST & FRIEDRICH, LLP, ONE SOUTH PINCKNEY STREET, P O BOX 1806, MADISON, WI, 53701, US

(73) Assignee: WOLFE RESEARCH PTY LTD, North Caulfield (AU)

(21) Appl. No.: 11/301,319

(22) Filed: Dec. 12, 2005

Related U.S. Application Data
(63) Continuation-in-part of application No. 09/786,252, filed on Aug. 13, 2001, now abandoned.

(30) Foreign Application Priority Data
Sep. 4, 1998 (AU) PP 5732
Sep. 22, 1998 (AU) PP 6056
Mar. 1, 1999 (AU) PP 8915

(51) Int. Cl. A61N 1/08; A61N 1/378

(52) U.S. Cl. 607/61

(57) ABSTRACT

There is provided a system for transmission of power and/or information between a first location external of a living body and a second position internal of the living body which comprises: (a) a primary controller (2) comprising a power source and a transmitter locatable at the first locations; and (b) an antenna (12) based device (10) locatable at the second position to receive an output from the transmitter, wherein the power source is adapted to emit high frequency electromagnetic radiation between 0.5 to 5 GHz. A medical appliance comprising a spring-based stent incorporating a monitoring device wherein the spring of the stent acts as the aerial for the monitoring device and wherein the medical appliance is capable of receiving electromagnetic radiation with a frequency between 0.5 to 5 GHz.

### CROSS-REFERENCE TO RELATED APPLICATIONS

This application is a continuation-in-part of U.S. application Ser. No. 09/786,252, filed Aug. 13, 2001, which is a national stage filing under 35 U.S.C. 371 of International Application No. PCT/AU99/00726, filed Sep. 3, 1999, which claims foreign priority benefits to Australian Application No. PP 5732, filed Sep. 4, 1998, Australian Application No. PP 6056, filed Sep. 22, 1998, and Australian Application No. PP 8915, filed Mar. 1, 1999. All of which are incorporated herein by reference.

### FIELD OF THE INVENTION

The invention relates to a system which facilitates monitoring, treatment and stimulation of a living body. More particularly, this system relies upon the use of electromagnetic waves as the means of transmission of energy and signals between a device implantable inside the living body and an external control device.

The invention, in a separate embodiment, also relates to a device that may be implanted inside the cardiovascular system so that properties of the environment within the body at which it is implanted may be monitored and a blood flow passage can be enlarged. The device is electrically powered and controlled by an external source of electromagnetic radiation and is not dependent upon the incorporation of a battery.

### BACKGROUND OF THE INVENTION

Whilst the following description is in terms of particular applications eg. muscle stimulation and the use of stents, it is to be understood that the invention has wider application.

A number of people all over the world lose their natural ability to control their muscle contraction and are thus physically disabled. Functional Electrical Stimulation (FES) is a technique which incorporates the stimulation of muscles for providing functionality to people suffering from neuromotor control disorders or have otherwise lost their natural ability to control and contract their muscles usefully. The disorder or loss of natural ability can arise through a range of causes, including disease, trauma or stroke.

FES devices can be classified into two categories-implants and external. External FES devices include simple devices such as those used to correct drop foot, and have been in use for a few decades. The implantable devices are relatively new and the first commercialisation of such a device took place in 1997.

In the present art, implantable devices consist of a controller and a set of up to 16 electrodes connected by wires which run inside the body (Memberg, Peckham, Keith, “A Surgically Implant Intramuscular Electrode for An Implantable Neuromuscular Stimulation System”, IEEE trans. Rehab. Eng, vol. 2, no. 2, June 1994, which is incorporated by reference herein). In this art, the device does not have any internal power source but it is powered by an oscillating magnetic field from the power source coupled with the secondary pick up coil implanted within the patient as a component of the device.

FES devices have been reported which provide this format (U.S. Pat. No. 5,358,514 to Schulman et al; Matjacic et al “Wireless Control of Functional Electrical Stimulation Systems”, PMnD: 9148704, UI: 97205715, which is incorporated by reference herein; Sawan, Hassouna et al “Stimulator Design and Subsequent Stimulation-Parameter Optimization for Controlling Micturition and Reducing Urethral Resistance”, IEEE trans. Rehab. Eng., vol. 4, no. 1, March 1996, which is incorporated by reference herein). In these devices, each of the muscle stimulating electrodes is addressable individually. The devices reported have employed frequencies of the magnetic field between 400 K Hz to 50 M Hz.

It is an intrinsic limitation of such magnetic technologies that the source of the oscillating magnetic field must be close to the pick up coil to efficiently transfer energy by inductive coupling of the magnetic field of the exciting source and the implanted magnetic coil receiver.

Some FES systems reported in the prior art provide the forward loop control for the muscles. Devices have been designed which record information from the extremities—either by recording neural activity or by using sensors (like pressure or vibration etc.) and feedback this information to the controller (Haugland, Hoffer et al “Skin Contact Force Information in Sensory Nerve Signals Recorded by Implanted Cuff Electrodes”, IEEE trans. Rehab. Eng., vol. 2, no. 1, March 1994, which is incorporated by reference herein). Some difficulties associated with these techniques are the invasive nature of their implementation and that further the information received is unnatural so the subjects have to learn to react to this information.

Available devices like the Drop Foot FES system automatically restore the gait of the subject and are not under the conscious control of the subject. FES systems like grasp control devices and other similar systems work under the linear control of the subject.

These latter devices have a number of drawbacks including the need for total visual attention of the subject which restricts the application of the device. Another drawback is the fact that these devices are not intelligent, unlike the body which has a Peripheral Neuromotor control mechanism which works along with the Central Neural System (CNS).

Thus subjects fitted with the FES devices have to use their CNS to monitor and control the muscle contraction.

A number of researchers have proposed systems which provide feedback to the subjects (Haughland, Hoffer et al, “Skin Contact Force Information in Sensory Nerve Signals Recorded by Implanted Cuff Electrodes”, IEEE trans. Rehab. Eng. vol. 2, no. 1, March 1994, which is incorporated by reference herein; Hoffer J D, “Closed Loop, Implanted Sensor, Functional Electrical Stimulation System for Partial Restoration of Motor Functions”, U.S. Pat. No. 4,750,499, which is incorporated by reference herein). These systems primarily utilise invasive methods like recording the neural activity, embedding sensors inside the body or fixing them on the surface of the body. These techniques are highly invasive and also restrictive to the subjects.

Another known method for muscle stimulation is multi-channel surface FES systems wherein the electrodes are supported on electrode trousers (Mayr, W et al “EMG-controlled adjustment and fatigue monitoring in multi-channel surface stimulators” Proceedings of the Second Annual IFESS Conference (IFESS'97) and Neural Prosthesis: Motor Systems 5 (NP'97) pages 13-14, which is incorporated by reference herein). Continued investigations, such as this, into external stimulation methods is a result of the above disadvantages of internal systems.

Accordingly, investigations were carried out to simplify these known highly invasive techniques. In particular, it was felt that if an alternative method could be developed to communicate between the external control and the internal devices, it may be possible to avoid or limit the use of wires in the body, in particular, to avoid the medically dangerous situation where wires penetrate a membrane such as the skin, intestinal walls or arterial walls. The desired alternative method would also permit monitoring, treatment and stimulation devices to be placed deeper and more locally to the area of interest in the body.

Various attempts to provide suitable systems and devices have been proposed. The following patents and patent applications disclose some of these attempts. Each of these patents and patent applications is incorporated by reference herein.

U.S. Pat. No. 5,314,458 (which is incorporated by reference herein)

The implantable microstimulator system employs a miniature ferrite-cored coil contained within a hermetically sealed housing to receive control signals and operating power from an RF telemetry system. The tiny coil receives the electromagnetic energy which is transmitted from a non-implantable transmitter which generates a code-modulated carrier.

Demodulator circuitry in the implantable microcircuit is employed to extract the control information, while applying the electromagnetic energy to power the electronic circuitry therein and charge a capacitor which will provide the electrical stimulation to the living being. The electrical stimulation is delivered by a stimulating electrode which has a waffle-like configuration whereby a plurality of iridium oxide electrode pads, coupled in parallel, so as to be characterised by a long effective edge distance, transfer the stimulating charge. The electrical components of the microstimulator are contained within a hermetically sealed housing formed of a glass capsule which is electrostatically bonded to a silicon substrate.

U.S. Pat. No. 5,735,887 (which is incorporated by reference herein)

The citation discloses an implantable, electrically operated medical device system comprising an implantable radio frequency receiver and an external radio frequency transmitter. The system is a closed-loop, inductively coupled radio frequency energy transfer system whereby the transmitted radio frequency power is adjusted up or down by the receiver as a function of received vs. required power, via commands up-linked by the receiver to the transmitter. The subcutaneous receiver incorporates the required faculties to autonomously control all stimulation parameters after it has been programmed only once.

The stimulation parameters controlled by the receiver are pulse amplitude, width and frequency, plus identification of the electrodes to be enabled and their respective polarity.

U.S. Pat. No. 5,769,875 (which is incorporated by reference herein)

This citation discloses a functional neuromuscular stimulation system. The system includes an implanted unit which is powered by the carrier frequency of the transmitted signal and stimulation pulse train decoders. The preferred embodiment uses a frequency of about 10 MHz.

European patent application no. 0 343 858 (which is incorporated by reference herein)

This citation discloses a telemetry system which comprises an implantable element having temperature dependent NMR properties, apparatus for applying a radio frequency field to the implantable element, and apparatus for sensing the temperature dependent NMR resonance response of the implantable element and for providing an output indication of temperature of the implantable element. The aim of the citation is to provide a wireless thermometry system useful in clinical hyperthermia. There is disclosure of the implantable elements including a rare earth metal which determines the resonance frequency to be used.

As an example, yttrium is said to resonate at approximately 53.578 MHz (0.0535 GHz) at 30 degrees C.

U.S. Pat. No. 3,662,758, which is incorporated by reference herein, discloses a telemetric system which operates at 350 kilocycles. A unit is implanted in the body which is powered by a source external of the body. The unit senses resistance between two electrodes inside the body and encodes the resistance as a frequency modulated signal which is then transmitted to a receiver outside of the body.

U.S. Pat. No. 3,727,616, which is incorporated by reference herein, discloses a telemetric system which operates at 100 KHz. A receiver totally implanted within a living body is inductively coupled by two associated receiving coils to a physically unattached external transmitter which transmits two signals of different frequencies to the receiver via two associated transmitting coils. One signal provides commands to the receiver and the other signal provides a power source.

U.S. Pat. No. 4,524,774, which is incorporated by reference herein, discloses a telemetric system which operates at 40.68 to 40.75 MHz. The system includes muscle potential sensors, muscle stimulators and a transmitter-receiver which receive and transmit signals via antennae without being wired to each other.

U.S. Pat. No. 4,102,344, which is incorporated by reference herein, discloses a telemetric system which operates at 300 KHz. The implantable unit has an energy storing device connected to electrodes under the control of a transistor which is normally maintained non-conductive as a result of the voltage drop across an impedance connected to the power supply so that each time the power supply is interrupted the transistor becomes conductive to discharge the energy storage device through the electrodes.

U.S. Pat. No. 4,494,950, which is incorporated by reference herein, discloses a telemetric system which operates at 10-50 KHz. The system consists of a multiplicity of separate modules which collectively perform a useful biomedical purpose; the modules communicating with each other without the use of interconnecting wires. The modules may be intracorporeal or extracorporeal.

Physiological sensor measurements sent from a first module caused a second module to perform some function in a closed loop manner.

U.S. Pat. No. 4,561,443, which is incorporated by reference herein, discloses a telemetric system which operates at frequencies of 51.2 KHz and 48.0 KHz. A two way coherent inductive communications link between an external transceiver and internal transceiver is disclosed which transmits digitally formatted data by frequency shift keying the inductive communications link. Further immediate verification of establishment of a reliable communications link is provided by determining the existence of frequency lock and bit phase lock between external and internal transceivers.

U.S. Pat. No. 4,628,933, which is incorporated by reference herein, discloses a visual prosthesis for implanting in an eye which is powered by telemetry. The prosthesis has a close-packed array of photosensitive devices on one surface thereof. There is disclosure of placing the transmitter about an eyeglass frame lens opening, so that the axis of the transmitting coil is oriented directly toward and in alignment with the axis of the coil.

U.S. Pat. No. 4,741,339, which is incorporated by reference herein, discloses a means to improve the electromagnetic coupling between the transmitter and receiver in a telemetric system by using a further coupling coil.

The system requires the transmitter and receiver to be close proximity.

U.S. Pat. No. 4,932,405, which is incorporated by reference herein, discloses a telemetric system which operates at a frequency between 100-500 KHz. The system disclosed is for stimulating a nerve or muscle fibre, especially a hearing nerve in the cochlea. The system includes an implant and electrode for stimulating the nerve which is connected to the implant. The system is powered by a small transformer wherein one coil is implanted and the other is external but in the vicinity of the implanted coil. For supplying information to the implant, infrared transmissions are used wherein the transmitter is provided adjacent to the skin and the receiver on the outside of the body.

U.S. Pat. No. 5,070,535, which is incorporated by reference herein, discloses a telemetric system designed to improve coupling efficiency between external transmitter and internal receiver. The citation requires that that receiver and transmitter be in very close proximity.

With the exception of U.S. Pat. No. 5,314,458, all of the devices in the above prior art rely on inductive coupling to transfer the energy and are therefore limited in their applications because they must be implanted close to the surface of the body in order to receive the signals from the primary control. The device in U.S. Pat. No. 5,314,458, which is incorporated by reference herein, receives electromagnetic radiation of a low frequency (that is, well below 0.5 GHz) and is a bulky device as a result of this low frequency.

There is also a well known art of medical appliances in the form of cylindrical shape with a wire cage called stents. These stent devices have been developed to enable cardiovascular surgeons and cardiologists to introduce these as part of their treatment to aid healing or relieve an obstruction. The stents are usually initially provided in a collapsed form on an inflatable support. In this form they are introduced into an appropriate blood vessel, such as the femoral artery near the groin, and carefully moved to the site of restricted blood flow.

The supporting balloon is then inflated so deforming the stent spring structure to press outwards into the wall of the blood vessel. The implanting apparatus and the inflatable support is then withdrawn, leaving the expanded stent to maintain the blood vessel open and allow improved blood flow.

It is further desired to combine a stent and a monitoring and/or stimulating device. In this way, it would be possible to continuously monitor the operation of the heart and provide information to assist preventative therapies to be adopted by a person.

One attempt to combine a stent with a monitoring device is described in PCT application WO 98/29030, which is incorporated by reference herein,. This application discloses a stent device which incorporates a device for measuring the fluid flow in the body of the person. The device for measuring blood flow transmits the measurement results to a receiver outside the body.

In one example of this, the stent has a resilient coil made of electrically-conductive material and coupled at both ends to circuitry associated with the flow parameter sensor and/or the transmitter. The energy source outside the body generates a time-varying magnetic field within the vicinity of the coil, which field is preferably aligned with a central axis thereof, this causing an electrical current to flow in the coil and provide energy to the flow parameter sensor and/or transmitter.

The system uses a magnetic coil to create a magnetic field so that a potential is created at right angles to the magnetic field and proportional to the flow rate. It is clear from the figures in this patent application that the size of the magnetic field is very large and it appears that the stent is also quite large and would be implanted in the aorta which has a diameter measured in cm (rather than mm as per other arteries and veins).

From the formula on page 20 of the citation, the frequency transmitted is about 0.8 MHz which is a low frequency. Again the low frequency gives rise to a bulky device. Further, the ECG recorder is separate from the stent and has sensor electrodes which are externally placed onto the skin rather than implanted.

Other examples of previous attempts to provide implantable devices are as follows.
U.S. Pat. No. 5,170,802, which is incorporated by reference herein, discloses an implantable electrode for location within a blood vessel. The electrode takes the form of a hollow, tubular metal structure expanded to contact with the inner walls of the blood vessel, ie a stent. However, the electrode is still physically connected to the electrical stimulator via an elongated insulated conductor (column 2, lines 37-38).
U.S. Pat. No. 5,411,535, which is incorporated by reference herein, discloses a cardiac pacemaker which wirelessly communicates with the electrodes. The implanted pacemaker body still requires a battery (power supply 159 at column 6, line 21) as does the electrodes (battery section 124 at column 6, lines 51-52). It is important to note that the instructions are transmitted within the body from the pacemaker to the electrode. The transmission does not need to deal with the change of medium from air through skin and fat and then tissue.
U.S. Pat. No. 5,967,986, which is incorporated by reference herein, discloses an endoluminal implant with fluid flow sensing capability. The implanted device is powered by a battery or a capacitor for storing energy (column 7 lines 43 to 44). The specification discloses use of a capacitor which relies on inductive coupling as explained by the need for the antenna to be implanted relatively close to the surface of the skin (column 12 lines 50-51) and FIGS. 7, 8 and 10. The description of FIGS. 7 and 8 (column 5 lines 6-13) expressly states that the external coil is electromagnetically couplied to the antenna.
U.S. Pat. No. 5,626,630, which is incorporated by reference herein, discloses a medical telemetry system using an implanted passive transponder. The transponder is specially designed to operate with an extremely low level of power so that it does not consume too much power from the implanted device which is battery powered (column 4 line 59 refers to the battery and column 9 lines 40-45 discuss recharging the battery).
U.S. Pat. No. 5,861,019, which is incorporated by reference herein, discloses an implantable medical device microstrip telemetry antenna which could be used with known medical devices such as known cardiac pacemakers which were battery operated.

Near Field and Far Field of Antennas

To understand how these prior devices operate it is important to understand the differences between near field and far field effects on antennas.

OET Bulletin 65 (FCC—Office of Engineering & Technology) (August 1997 edition), which is incorporated by reference herein, defines near field and far field as follows:
“Far-field” refers to that region of the field of an antenna where the angular field distribution is essentially independent of the distance from the antenna. In this region, the field has a predominantly plane-wave character, ie locally uniform distribution of electric field strength and magnetic field strength in planes transverse to the direction of propagation.
“Near field” refers to the region generally in proximity to an antenna or other radiating structure, in which the electric and magnetic fields do not have a substantially plane-wave character but vary constantly from point to point. The near field region is further subdivided into the reactive near-field region, which is closest to the radiating structure and that contains most or nearly all of the stored energy, and the radiating near field region where the radiation field predominates over the reactive field, but lacks substantial plane-wave character and is complicated in structure. For most antennas, the outer boundary of the reactive near field region is commonly taken to exist at a distance of one-half wavelength from the antenna surface.

An electromagnetic wave comprises both a time varying electric field and a time varying magnetic field. These fields are assumed to vary sinusoidally in time at a frequency f cycles per second. The wavelength λ of the electromagnetic wave can be defined as λ=c/f where c is the propagation velocity (3.0×10⁸ m/s in free space). The relation between the wavelength λ and the dimensions L of field generating and field absorbing structures or their distances is extremely important. When the dimensions L<<λ, the wave nature of electromagnetic energy can be essentially disregarded. The field amplitude typically decreases as l/r² or l/r³ where r is the distance from the source.

In the near field situation, the electronic and magnetic fields are effectively separable because of the relatively low frequencies involved in this mechanism. The energy transfer is achieved by either electric field interaction (E>>H) or primarily by magnetic (H>>E). E is the electric field intensity and H is the magnetic field intensity. In the near-field region, for energy to be transferred, the transmitting and receiving antenna is required to be placed in very close proximity to each other, so that either the E or H field couples from the transmitter to the receiver. Energy transfer by this means is achieved through coupling of the H field from the transmitter to the receiving coil, which is tuned to the transmitter frequency by a parallel capacitance.

For example, U.S. Pat. No. 6210347, which is incorporated by reference herein, discloses a Food Intake Restriction Device which uses energy in the frequency of 100 kHz to 1 GHz with the external signal transmitting antenna positioned close to the a signal receiving antenna implanted close to the skin where the antennae are coils. These details indicate that the device relies on a near field energy transmission mechanism.

Where the dimension L>>λ, then the electromagnetic waves propagate the energy and the field amplitude decreases as l/r where r is the distance from the source. To have an effective energy transfer in this far field situation, the frequencies must be higher than those used for the near field situation. For example, at radio or microwave frequencies, the ratio between E and H is equal to the wave impedance (approximately 377Ω for free space). In this region, the energy is transmitted as an electromagnetic wave and can propagate over much larger distances, allowing a greater separation between the transmitting and receiving antennae.

In summary, the difference between the near field region and the far field region is the nature of the energy propagation.

In the near field region, the fields are bound to the transmitter and rapidly decay in amplitude as you move away from the source. Transfer of energy can only be achieved if the receiving antenna is placed sufficiently close to the source to enable coupling to either the E or the H field from the source.

In the far field region, the source antenna radiates the energy in the form of an electromagnetic wave. This wave can then propagate over distance and through materials such as air and tissue. The receiving antenna therefore does not have to be placed in close proximity to the transmitting antenna provided there is sufficient power being transmitted.

The extent of the near and far field regions can be illustrated in the following expression: If an antenna whose largest dimension D is considered, then the near field region encompasses distances from the antenna surface up to 0.62√(D³/λ). At distances greater than this, only the radiated electromagnetic field exists and this defines the far field region.

There is a need for an effective medical implant system which operates in the far field region and this is what the invention addresses.

There is also a need for improvements with respect to the size of the antenna that is required so that smaller implantable devices can be developed.

In developing the current system, the selection of the suitable frequency range which would allow far field distances also needed to meet the following parameters:
Does the frequency used allow sufficiently low loss electromagnetic wave propagation through tissue to maintain the effectiveness of the system?
Are the antenna sizes acceptable for an implantable device (increasing the frequency reduces the physical size of the antenna)?
Can a suitable range of implant depths be used (implant depth depends upon the antenna gain, transmitter power and signal loss through the tissue which are all impacted by the frequency used)?

The electrical properties of tissues can be described by the dielectric permittivity (εᵣε₀) and the conductivity (σ). Both the dielectric relative permitivity (εᵣ) and the conductivity (σ) for tissues vary with frequency. Where the signal frequency is such that σ>>(2πf)εᵣε₀, the tissue may be regarded as a good conductor and an electromagnetic wave will not easily propagate through it. For a frequency where a does not meet the inequality, then the tissue may be regarded as a dielectric and an electromagnetic wave can propagate through it, although undergoing some reduction in amplitude due to the loss in the tissue. Typically at low frequencies the relationship between σ and εᵣε₀ is such that the tissue can be regarded as a conductor.

The power density at the skin surface must not exceed the level which could damage skin tissue, whilst meeting the requirements to provide power the antenna in order to drive the activated muscle stimulation implant. The power density impinging on a receiving antenna can be calculated from the following expression: Sᵣ=Pᵣ/Aₑ where Sᵣ is the received power density in mW/cm², Pᵣ is the received power in mW and Aₑ is the effective aperture of the antenna in cm². The effective aperture represents the capture area of the antenna when illuminated with an electromagnetic wave.

If the required received power for operation of remote activated muscle stimulation is defined to be up to +5 dBm, for example. The received power in mW to achieve this required power is:
Pᵣ=10^{(+5/10)}=3.162 mW.

The power density Sᵣ at the surface of the receiving antenna can then be calculated from
Sᵣ=(3.162/Aₑ)mW/cm².

For the receiving antenna implanted under a layer of fatty tissue which has a loss of L dB, then the power density at the surface of the fatty tissue (ie the skin) will be:
Sₛ=(10^{(L/10)})(3.162/Aₑ)mW/cm².

The present invention addresses this need for an effective medical implant system which operates using a suitably sized antenna.

### DESCRIPTION OF THE INVENTION

It has now been found that there is a range of appropriate frequencies within the electromagnetic spectrum where the radiation travels through space outside the body and then penetrates through the skin and fat continuing on to penetrate into flesh effectively. This permits the transmitter to be separated a convenient distance from the receiver that is many times the separation permitted by known coupled magnetic fields.

Accordingly, there is provided a system for transmission of power and/or information between a first location external of a living body and a second position internal of the living body which comprises:
(a) a primary controller comprising a power source and a transmitter locatable at the first location; and
(b) an antenna based device locatable at the second position to receive an output from the 1 5 transmitter,

wherein the power source is adapted to emit high frequency electromagnetic radiation between 0.5 to 5 GHz.

In particular, the preferred range of high frequency electromagnetic radiation is 0.8 to 3.5 GHz. In one preferred embodiment the high frequency electromagnetic radiation is 0.8 to 2.5 GHz. In another embodiment, the preferred range is 1.5 to 3.5 GHz. This high frequency electromagnetic radiation is receivable by the antenna on the implanted device and used as a source of electrical energy to power the device as well as being capable of carrying an information signal to operate the implanted device.

The preferred frequency of operation will be a compromise between using higher frequencies to enable the use of a smaller antenna but at the same time using lower frequencies to reduce tissue attenuation and other factors.

A person skilled in the art will know the type of hardware which may be used to construct an implanted device capable of being powered by the transmission of high frequency electromagnetic radiation between 0.5 to 5 GHz. Typically, the hardware will include a capacitor capable of receiving remote power and storing it to provide a continuous energy source. Capacitors are electric circuit elements used to store electrical charge temporarily. The electrical charge is stored as electrostatic charge and can be released quickly and evenly.

A person skilled in the art will understand that as technology improves capacitors that these improved capacitors could be used in an implanted device according to the present invention. A person skilled in the art will know that capacitors are different to batteries. Batteries are devices which generate electrical current by converting chemical energy to electrical energy. Since batteries rely on a conversion of chemical energy to electrical energy, they are slower at releasing energy than capacitors.

It was surprisingly found that the use of high frequency electromagnetic radiation between 0.5 to 5 GHz allows significant spatial separation of the primary controller and the implanted device. As such it potentially avoids wires to implanted devices such as stimulating electrodes and permits a number of devices to be implanted deep in the body. That is, the present invention enables the use of a single primary controller to power and control several implanted devices within a body.

Further, the use of radiation at this frequency removes the need to use coils in the antenna based device because there is no inductive coupling. Inductive coupling requires that the two coil antennas be carefully aligned and also in close proximity, ie 1 cm or less. The present invention relies on far field energy transmission and therefore it is possible for there to be a significant distance between the primary controller and the body containing the implanted device. The separation distance possible between the primary controller and the implanted device is dependent on transmitter power and the gain of its associated antenna as well as the efficiency of the implanted device(s). Preferably, the distance is up to 12 meters. Typically, the separation distance is at least about 4 meters. This distance is important as it enables the use of a number of implants controlled by a single primary controller.

It was also found that these high frequencies permitted the use of antennae that were small enough to conveniently implant but still to permit significant penetration of the electromagnetic energy into the body. The antenna format could be, for example, a simple dipole, a loop with or without crenellations, or a microstrip antenna including slot and patch formats. The preferred alternative is a planar omnidirectional format that is integrated into the construction of the device. For example, the antenna may be a 1 cm² patch antenna or a 3 to 5 mm loop antenna. The smaller the antenna the higher the frequency which is used. Summarising the above benefits, the present invention allows the use of:
a single primary controller;
several implanted devices in a single body powered and controlled by the single primary controller;
the primary controller placed at a significant distance from the body;
the implanted devices being small and having small antennae;
the implanted devices able to be implanted deep into the body, even within the vessels of the heart (ie, not close to the surface of the skin);
no separate battery activation of the implanted devices is needed; and
the implanted devices able to be implanted all over the body and not required to be in close proximity to each other.

That is, the present invention enables truly remote operation of the implanted devices.

Preferably, the primary controller may comprise other devices, for example, a receiver to receive data from the implanted device. In this respect, the implanted device may be used to sense properties of its environment and then transmit such data as electromagnetic radiation to the receiver.

Accordingly, it is preferred for the antenna based device to comprise means to monitor predetermined conditions adjacent the antenna based device and to emit signals representative of one or more of these conditions to be received by the primary controller.

By way of illustration only, the device may:

(a) measure the activity of the heart in terms of a electrocardiogram; and

(b) transmit this information to the primary controller.

In this way, it is possible to continuously monitor the operation of the heart and provide information to assist preventative therapies to be adopted by a person.

It is also preferred that the antenna based device may itself be a medical appliance which could operate in response to the transmitted signal. For example, the antenna based device could be a stent which is spring based where the spring acts as the antenna. This device may also be used to derive the data needed to register an electrocardiogram as described above.

Another example of a suitable antenna based device is a wireless electrode which communicates with an external pacemaker. This device would be useful for use after heart surgery and provide improved patient comfort as the frequency range of the invention enables the external pacemaker to be positioned in a room near the bed and the patient's movement would not be restricted by wires or the need to have the external primary controller placed close to their body.

Another example of a use for an implanted antenna device according to the invention is electrodes for implant into the brain as stimulators to suppress epilepsy or Parkinson's tremors or as sensors to remotely control motor function.

A further example of a use for an implantable antenna device is to assist with artificial sight. Current devices for artificial sight are bulky and resemble a motorcycling helmet. The present invention would enable the development of a more practical sized device for widespread use.

According to yet another preferred form, the antenna based device may comprise means to generate pulses of current. By way of illustration only, the device may:
(a) take the transmitted signal, send out pulses for muscle stimulation as specified by the signal regulating commencement time, pulse width, pulse frequency and number of pulses;
(b) measure electrocardiogram (ECG), pCa, glucose, p02, pNa, electromyogram (EMG), pH, muscle dimensions and transmit this data to the primary controller;
(c) be a combination of features (a) and (b);
(d) measure the Electroencephalogram inside a cranium to detect abnormal brain conditions such as epilepsy and transmit a signal to the primary controller to activate an alarm;
(e) send out suitable pulses in response to the condition sensed in (d) to trip the brain action back into normal activity.

According to a second aspect of the invention, there is provided a method for transmitting power and/or information between a first location external of a living body at which a primary controller comprising a power source and a transmitter is located, and a second location inside the living body at which an antenna based device is located, the method comprises the steps of:
(a) generating high frequency electromagnetic radiation between 0.5 to 5 GHz from the power source and emitting that radiation from the transmitter of the primary controller; and
(b) receiving the radiation at the antenna based device.

In particular, the preferred range of the high frequency electromagnetic radiation is 0.8 to 2.5 GHz.

Preferably, the method comprises the further steps of:
(c) powering the antenna based device with the radiation; and/or
(d) causing the antenna based device to generate and emit pulses of current; and/or
(e) monitoring predetermined conditions adjacent to the antenna based device and emitting signals representative of one or more of these conditions to be received by the primary controller.

It has also been found that a stent and a monitoring device may be combined into a single unit thereby achieving two objectives with one operation. Further, the combined device resembles a standard stent, and therefore may be implanted into the patient using the same procedure as for a standard stent.

According to a third aspect of the invention, a medical appliance is provided which comprises a spring-based stent incorporating a monitoring device wherein the spring of the stent acts as the aerial for the monitoring device and wherein the medical appliance is capable of receiving electromagnetic radiation with a frequency between 0.5 to 5 GHz.

Preferably, the monitoring device is located in the support of the stent. Preferably, the monitoring device works in conjunction with a primary controller. The monitoring device will preferably comprise means to monitor predetermined conditions in the vicinity the medical appliance and means to emit signals representative of one or more of these conditions to be received by the primary controller.

Preferably, the primary controller is separate and located outside the body in which the stent is implanted. Preferably, the primary controller is adapted to emit high frequency electromagnetic radiation between 0.5 to 5 GHz. This is particularly useful for deep implants. Preferably, the primary controller is a power source for the monitoring device.

In situations where it is difficult to communicate directly with the medical appliance, a second intermediate implant may be necessary which is closer to the skin surface and which can relay the power and instructions from the primary controller to the medical appliance.

### DETAILED DESCRIPTION OF THE INVENTION

Whilst the following discussion is in terms of using the above system and method for stimulation purposes, it will be understood from the discussion above that the invention is not so limited. The invention provides a system of interaction between a location outside the living body and a location inside the living body which permits power and/or information to flow therebetween. The nature of the information and use of power will depend upon the antennae based device implanted in the living body.

In a first example of the invention, there is provided a stimulation device for providing artificial electrical stimulation comprising a receiver antenna for receiving electromagnetic radiation ranging from between 0.5 to 5 GHz from a primary controller, a supply circuit for deriving electrical energy from the received electromagnetic radiation, an isolating circuit for isolating data signals from the received electromagnetic radiation, a pulse generator for generating electrical pulses according to the data signals utilising the electrical energy from the supply circuit, and a stimulating electrode for outputting the electrical pulses from the pulse generator.

In other words, this stimulation device comprises an antenna for receiving electromagnetic radiation in the range between 0.5 to 5 GHz from a primary controller and converting it to an oscillating current, a converter for converting the oscillating current to an electrical supply suitable to provide power for the device, an isolating circuit for separating a data signal from the oscillating current, and a pulse generator activated according to the data signal to provide electrical stimulation pulses using said electrical supply power The stimulation device may therefore be at least substantially encapsulated in a biocompatible material, such as a suitable epoxy, silicone polymer, “diamond” coating or the like. The stimulating electrode can be constructed from a suitable biocompatible conductive material, such as titanium, surgical stainless steel, gold, osmium, iridium and platinum. The components of the stimulation device may be contained in a single substantially encapsulated unit for ease of surgical implantation, however it is possible that the antenna and/or electrode be separate and connected to the remainder of the device by way of a short wire, for example. This construction may be desirable where the site to be stimulated by the device (i.e. the desired position of the electrode) is located relatively deep within the subject tissue. The concept of the invention would permit the antenna to be near the tissue surface for reduced attenuation of the electromagnetic radiation received at the antenna. It may additionally be desirable to provide a coating or patch of an anti reflection material on the tissue surface over the antenna to further reduce electromagnetic radiation signal attenuation.

In another example of the invention, a plurality of stimulation devices are used and are responsive to signals from a single primary controller. In this case, it is desirable for each stimulation device, or groups of stimulation devices, to be selectively actuated by the received data signals. Accordingly, the isolating circuit or pulse generator is preferably constructed to be addressable by certain data signals, such that stimulation pulses are only generated if a certain form of data signal is received from the primary controller. For example, the stimulation device can be constructed to decode modulated digital codes and compared with predetermined codes to ascertain whether that particular device is being addressed. Alternatively, a form of frequency signal coding can be used, and the isolating circuit adapted to isolate only the data signals intended for that device. Other data encoded in the data signals can be utilised by the pulse generator to control the characteristics of electrical pulses generated, such as pulse shape, magnitude, duration and frequency.

Most patients require several devices to stimulate various muscles and sense their condition and this may be achieved by the central primary controller sending the signals that contain addresses of the particular electrodes to be activated or the transmitted data contains related addresses.

For example, this invention allows the patient to have the many electrodes required to stimulate walking without the fragile wires crossing joints.

According to a further embodiment of the invention, there is provided an artificial muscle stimulation system comprising at least one stimulating electrode for providing artificial electrical stimulation to a muscle under control of a primary controller capable of transmitting high frequency electromagnetic radiation between 0.5 to 5 GHz, an EMG sensor for measuring EMG signals from the muscle during stimulation, a neural network processor coupled to receive the measured EMG signals to extract information regarding force of contraction and fatigue of the muscle, and wherein the primary controller is coupled to an output of the neural network processor to control said artificial electrical stimulation based on said extracted information.

It has been discovered that particular muscles rapidly tire if stimulated incorrectly but this may be avoided if the muscle is stimulated in different regions or less frequently. The art of stimulating muscles requires careful monitoring of several aspects to avoid tiring. It has been discovered that the EMG of the working muscle can be used to characterize the onset of tiring as can extension over time, pressure of the muscle during contraction and the pH of the tissue of the muscle. The primary controller then varies the stimulation to accommodate the tiring muscle.

It is well known that muscle fatigue is associated with the production of lactic acid rather than carbon dioxide and this is monitored by measuring the pH and p02 of the muscle.

Similarly, with the medical appliance of the third aspect of the invention, it is valuable to measure the glucose concentration and p02 as the onset of aschemia in a diabetic is indicated when the glucose is high and the p02 is low. This indication with the ECG is useful for diagnosis of a potentially dangerous condition of the patient.

In an embodiment of the third aspect of the invention, the wire spring structure of a stent performs the known basic function of expanding blood vessels, and can also conduct electrical signals and thereby act as the antenna for receiving electromagnetic energy. The small diameter of blood vessels and the reduction in wavelength caused by the high permittivity of blood and blood vessel wall, requires that the frequency of the electromagnetic radiation be greater than 0.5 GHz. It has been surprisingly found that electromagnetic radiation up to for example, a frequency of 1.7 GHz can be usefully transmitted to an antenna that is implanted inside a blood vessel and immersed in blood.

The high frequency electromagnetic radiation causes a typical oscillating current in the wire of the stent and this current may be modified by designing the inductance and capacitance of the wire structure to induce resonance. The resulting current is rectified and used to power the monitoring device.

The direct current is then used to charge either a capacitor. Typically, the circuit would be a low power microprocessor with both A/D (“analogue/digital”) inputs and output drivers suitable for generating the pulse train to be applied to the antenna for transmission out of the body. For simple versions of the technology, the function of the microprocessor would be replaced by discrete or partially integrated circuits that perform the function of processing the signals from the sensor, analysing the signal then transmitting the alarm signal.

In this arrangement, the electronics are typically used to monitor the electrocardiogram but may also monitor pH, blood flow, pCa and other metabolites. The device also has provision to transmit signals out of the body, typically to give an alarm for an abnormal condition.

In one practical form of the invention, the stent is configured as stiff hoops to expand blood vessels but the surgical procedure requires that they be implanted in a collapsed form. Each hoop is pleated with the pleats roughly sinusoidal so that the amplitude of the sinusoid is normal to the plane of the hoop so making the sinusoidal in the same cylindrical plane as the wall of the blood vessel in which is implanted. The pleating is controlled in amplitude and number of pleats to give a radiation impedance for the antenna similar to the space impedance of the body environment. Similarly, the pleating also gives some control over the inductance and capacitance of the antenna considered as a resonant tank circuit together with the characteristics of the rectifier.

### EXAMPLES

The invention will now be further explained and illustrated by the following non-limiting examples.

Development of an antenna of an appropriate size was investigated, especially for operation in the frequency range from 0.5 to 5 GHz. Over this frequency range, the εᵣ is fairly constant with frequency (around 4.5 to 7.5 depending on the tissue sample) and the σ monotonically increases with increasing frequency from about 0.03 to 0.4 Siemens/m.

Examples 1 to 4 investigate the fabrication of antennae which will receive radiation with a frequency between 0.5-5 GHz.

Investigations showed that human tissue properties are very attractive for relatively low-loss electromagnetic wave propagation over the frequency range of 0.5 to 2.5 GHz. This frequency range also allows the use of suitable sized antennae. The usefulness of this frequency range has been demonstrated by the experiments.

The experiments show that signal propagation is possible through tissue at these frequencies and capable of delivering a suitable received power level to an implant without requiring dangerously high power densities at the skin surface. These experiments were conducted with a separation between the transmitting and receiving antennae which was much greater than the distances specified in the prior art and which clearly puts the receiving antenna of 50 cm in the far field region relative to the transmitting antenna.

Investigations show that selection of a frequency within the operating range of 500 MHz to 2.5 GHz is a trade-off between:
Antenna size (decreases with increasing frequency),
Transmitter power (increases with increasing frequency due to the increasing loss in tissue), and
Allowable implant depth (decreases with increasing frequency due to the increasing loss in the tissue and the limitation set on the transmitter power based on the safe maximum power density and the skin surface).

For the patch antennas used, the effective aperture of the antennae can be estimated by equating it to the area of the patch.

| Antenna | Dimensions | Effective aperture (cm²) |
|---|---|---|
| 1 | 17 mm × 17 mm | 2.89 |
| 2 | 29 mm × 29 mm | 8.41 |
| 3 | 33 mm × 33 mm | 10.89 |
| 4 | 50 mm × 60 mm | 36 |

Various measurements of signal path loss between a transmitting antenna and the designed patch antennae were undertaken for situations where the antenna was located behind a single fat layer of 1 cm thickness and two fat layers of 2 cm thickness total. The measurements were performed for the antennae in Examples 1 to 4 below and the results are depicted in FIGS. 8 and 9.

The predicted power densities were well below the safety limit for human skin of I to 5 mW/cm² (limits in OET Bulletin 65, (FCC—Office of Engineering & Technology) August 1997 edition, which is incorporated by reference herein). The power densities would be even lower for the case of an implant transmitting a return signal.

### Example 1

A microwave patch antenna 17 by 17 mm area with a separating dielectric of relative permittivity 10.2 and 1.905 mm thickness was fabricated, coated with Dow Corning Silicone polymer and placed inside a moist piece of fatty tissue/skin at a depth of 10 mm.

The antenna was excited with electromagnetic radiation of 500 milliwatts from a transmitter and the frequency varied near 2.5 GHz to establish the optimum resonant frequency. The power received at the antenna was measured using a microwave power meter when the transmitter was at 12 and 50 cm and found to be 10 mW and 1.6 mW and at 12 cm the output of the antenna was recitified with a full wave bridge and showed a voltage of 2.5 volts.

### Example 2

A microwave patch antenna 29 by 29 mm area with a separating dielectric of relative permittivity 10.2 and 1.905 mm thickness was fabricated, coated with Dow Corning Silicone polymer and placed inside a moist piece of fatty tissue/skin at a depth of 10 mm.

The antenna was excited with electromagnetic radiation of 500 milliwatts from a transmitter and the frequency varied near 1.5 GHz to establish the optimum resonant frequency. The power received at the antenna was measured using a microwave power meter when the transmitter was at 12 and 50 cm and found to be 25 mW and 3.2 mW and at 12cm the output of the antenna was rectified with a full wave bridge and showed a voltage of 2.3 volts. The thickness of the fatty tissue was then increased to 20mm and the test repeated and showed at 50 cm a power output of 2.5 mW and at 100 cm a power output of 0.4 mW.

### Example 3

A microwave patch antenna 33 by 33 mm area with a separating dielectric of relative permittivity 2.2 and 1.58 mm thickness was fabricated, coated with Dow Corning Silicone polymer and placed inside a moist piece of fatty tissue/skin at a depth of 10 mm. The antenna was excited with electromagnetic radiation of 500 milliwatts from a transmitter and the frequency varied near 2.5 GHz to establish the optimum resonant frequency. The power received at the antenna was measured using a microwave power meter when the transmitter was at 12 and 50 cm and found to be 10 mW and 0.8 mW and at 12 cm the output of the antenna was rectified with a full wave bridge and showed a voltage of 2.6 volts.

### Example 4

A microwave patch antenna 60 by 60 mm area with a separating dielectric of relative permittivity 2.2 and 1.56 mm thickness was fabricated, coated with Dow Corning Silicone polymer and placed inside a moist piece of fatty tissue/skin at a depth of 10 mm. The antenna was excited with electromagnetic radiation of 500 milliwatts from a transmitter and the frequency varied near 1.5 GHz to establish the optimum resonant frequency. The power received at the antenna was measured using a microwave power meter when the transmitter was at 12,50 and 100 cm and found to be 25 mW, 6.3 mW and 0.8 mW and at 12 cm the output of the antenna was rectified with a full wave bridge and showed a voltage of 2.8 volts. The thickness of the fatty tissue was then increased to 20mm and the test repeated and showed at 50 cm a power output of 3.2 mW and at 100 cm a power output of 0.25 mW.

Summary

Examples 1 to 4 illustrate that radiation with a frequency between 0.5-2.5 GHz can be used to generate power in an antenna based device without the need for inductive coupling.

In Examples 5 to 8 investigations were conducted into the fabrication of devices which could be used in a medical device according to the third aspect of the invention.

### Example 5

Surgical stainless steel wire 316LVM and diameter 0.0059 in. was pleated with a sinusoid of amplitude 0.039 in. giving five cycles in 0.83 in. This planar structure was then bent to form a hoop and attached to a Schottky diode and measuring apparatus. The entire assembly was coated with a biodegradable resin such as silicone polymer to provide electrical insulation from the biological fluids.

The device was implanted in the artery of a bovine liver and irrigated with heparinised blood. The entire assembly was then transferred to a chamber for testing microwave transmitters and irradiated with electromagnetic energy that was varied in frequency between 0.5 GHz and 2 GHz and the energy received monitored. This test showed satisfactory energy was received up to a frequency of 1300 MHz, with several peaks including 850 MHz, and gave an output of 1.5 volts and 400 microwatts when immersed in blood and excited.

It would be understood that a variety of types of wire are useful including titanium and metals in the platinum group, and the wire may have coatings to reduce energy loss by conduction through the body electrolyte and improve the acceptance of the device by the body immune system. Similarly, a wide variety of stent configurations are workable and most of these can be formed into useful antennas.

### Example 6

The antenna was constructed with the support of the sinusoidal (or crenellated) loop, supported by an extension of the ends of the loop, at right angles to the main plane of the loop, as parallel wires also contained in the silicone polymer create a capacitance in series with the loop.

The length of the parallel wires was made in 3 mm so that when the self inductance of the loop generates an impedance to the oscillating current in the loop wire, it is matched by the impedance of the capacitance and the assembly then causes a tank circuit oscillation with a large increase in available voltage.

The device was tested with radiation at 0.86 GHz and gave 2 volts and 800 microwatts when immersed in blood medium.

### Example 7

The antenna of Example 6 was used to power a Sharp SM5K3 microprocessor so that the incorporated analog to digital (A/D) converter could be used to input the low frequency signal of an ECG which was simulated on a 1 Hz triangle wave in the blood medium. The output of the microprocessor generated a one bit signal when it had power and had detected the simple signal.

### Example 8

The second A/D converter of the microprocessor was used to measure pH by incorporating a miniature pH glass electrode and silver/silver chloride reference electrode. The pH was changed by addition of acid to the blood medium and the microprocessor registered this change by an output of changing output.

Summary

Examples 5 to 8 illustrate that a medical appliance can be fabricated which will receive radiation with a frequency between 0.5-5 GHz.

### DESCRIPTION OF THE DRAWINGS

The invention will now be further illustrated with reference to the accompanying drawings in which:

FIG. 1 is functional block diagram of a wireless electrical muscle stimulation system embodying the first two aspects of the invention;

FIG. 2 is a functional block diagram of a receiver and activator for a wireless FES system;

FIG. 3 is a block diagram of a second embodiment of the first two aspects of the invention;

FIG. 4 is a block diagram showing the construction of a digital form of the receiver activator;

FIG. 5 is a block diagram of a system for providing feedback for artificial stimulation;

FIG. 6 is a conceptual view of a third embodiment of the first two aspects of the invention;

FIG. 7 is a side perspective view of an embodiment of the third aspect of the invention.

FIG. 8 is Measured loss through fat layers for antennae 1 to 4.

FIG. 9 is power density at the skin surface for a received power of +5 dBm for antennae 1 to 4.

FIG. 10 is an illustration of the set up used in Example 9.

FIG. 11 is a photo of a patient using a device according to the prior art.

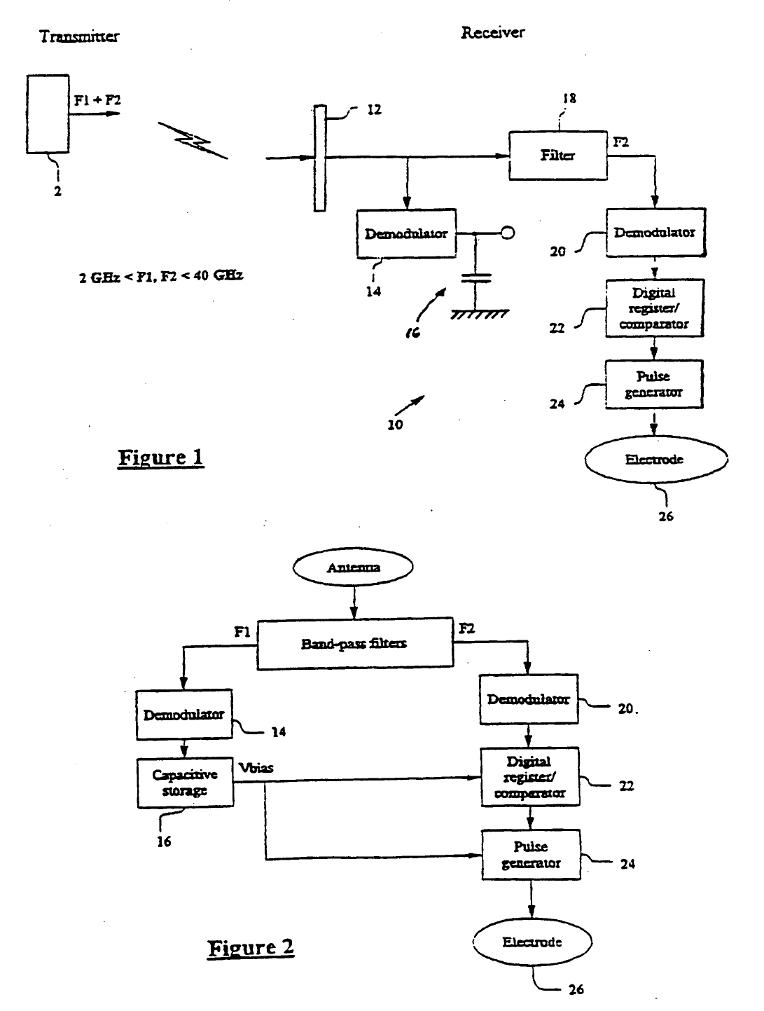

As introductory comment to the description of the drawings in FIGS. 1 to 6, the antenna based device is a receiver and addressable activating device to enable electrical stimulation of muscles (skeletal, smooth or cardiac) as described below. This receiver is constructed to enable it to be implantable within the body of the subject, and in practice a plurality of receivers would be implanted at different locations in the body to stimulate different muscles. The receiver derives its energy for operation from electromagnetic radiation emanating from a primary controller and not from a battery. The primary controller also provides, by way of the electromagnetic signals having a frequency between 0.5 to 5 GHz, commands to control the receiver and activator so as to produce appropriate electrical stimulation signals to the muscle.

To enable a wireless FES system to operate with multiple receivers/activators stimulating different muscles and to be controlled by a single primary controller, it must be able to control each receiver/activator individually. To achieve this, each receiver can be constructed to respond only to a certain form of signal issued from the transmitter. There are various ways in which that can be implemented, comprising a digital addressing scheme and a frequency coded addressing scheme. Because the system is wireless, and both power and control signals are transmitted from the primary controller to the multiple receivers by way of the stated electromagnetic radiation, numerous receiver/activators can be controlled using a single primary controller without the difficulties associated with implanted or even external wiring, such as wires passing through jointed areas in the body.

Each receiver comprises an antenna, also implanted, tuned to receive the electromagnetic radiation from a primary controller which may be worn on or about the body of the subject. The receiver antenna does not need to be specifically lined up with the primary controller.

As indicated, the high frequency electromagnetic signals are in the range of 0.5 to 5 GHZ.

A portion of the signal energy is utilised to provide electrical power to the activator circuitry, and another portion of the signal is decoded to provide control information such as the address of the receiver/activator and the shape and size of pulse to be provided at the output electrode.

This receiver/activator device is preferably encapsulated using a biocompatible resin such as silicone. The output of the activator is a stimulating electrode which is preferably constructed of titanium or a similar biocompatible conductive material. The electrodes are self attaching or may be sutured to the muscle, and can be constructed of a form which is known in the art. The size of each output electrode may be of the order of 2 mm to 20 mm.

If the muscle to be stimulated is located relatively deep inside the body, the receiving portion of the device, comprising the antenna, can be located near the surface and provided with a short wire link to the activating site, however it is preferable to select a frequency of the electromagnetic radiation that permits the entire device to be close to the nerve site being stimulated using electrodes on the surface of the device or very short leads to the stimulating electrodes It may be advantageous to provide a coating or patch of an anti reflection material (suitable for the electromagnetic frequency utilised for communication between the transmitter and receiver) positioned on the skin of the subject where the receiver is located, if it is desirable to reduce the required level of radiated energy such as for the abdomen area.

Turning to the drawings, FIG. 1 is a functional block diagram of a primary controller 2 and receiver 10 system. The receiver and activator device 10 is also illustrated in block diagram form in FIG. 2. The device 10 includes a dipole antenna 12 which is constructed to receive electromagnetic signals radiated from the primary controller 2. Data signals and power is transmitted by the primary controller 2 at frequencies which are in the range of 0.5 to 5 GHz. The dipole antenna 12 can be constructed from a suitable conductive material, such as titanium, or an integrated circuit die, and may have the dimensions of, for example, 8 mm length, 4 mm width and 2 mm depth. The signals received by the antenna are passed to passive demodulating circuitry 14 of known construction. Signals of one frequency, F1, are thereby demodulated to provide an electrical power source for the activator circuitry 22, 24, 26. The electrical power provided by the output of demodulator 14 is used to charge the capacitive storage element 16.

A second frequency, F2, produced by the primary controller 2 is the carrier frequency which carries information responsible for addressing and controlling the specific receiver/activator device 10. Passive filtering circuitry 18 of conventional design can be used to isolate the control signals at carrier frequency F2, which are then demodulated. The control signals provided by the output of the demodulator 20 are passed to the activator circuitry 22,24,26.

The activator circuitry portion of the device 10 comprises a digital register and comparator 22 which is able to decode the address portion of the transmitted data. The address is provided to enable selection of one single activation device or a group of devices, and a given activator may be required to be able to decode more than one address (eg one address for the particular device itself and one address for each of group of devices it may belong to). The second burst of pulses is decoded by the devices selected according to the address information, and this provides the information for that device regarding the shape and size of the pulse to be generated at the stimulating electrode. The pulse according to the received data is thus generated by the pulse generator 24, which can also be of conventional form, appears at the electrode plate 26 to stimulate the tissue it is embedded in. The electrode plate may be physically next to the rest of the receiver/activator device 10, or may be a short distance away and coupled thereto by an insulated multistrand stainless steel wire, for example. The device 10 is designed to deliver a variable current from the output electrode 26. This provides the flexibility for use in various different applications. The shape and rate of the train of pulses generated by the pulse generator is dependent on the transmitted signals, and can be dynamically controlled by the primary controller 2 to meet the muscle recruitment requirements. This flexibility is useful in order to be able to have a control over the recruitment of motor units. This is a feature that the existing stimulators have not been able to offer.

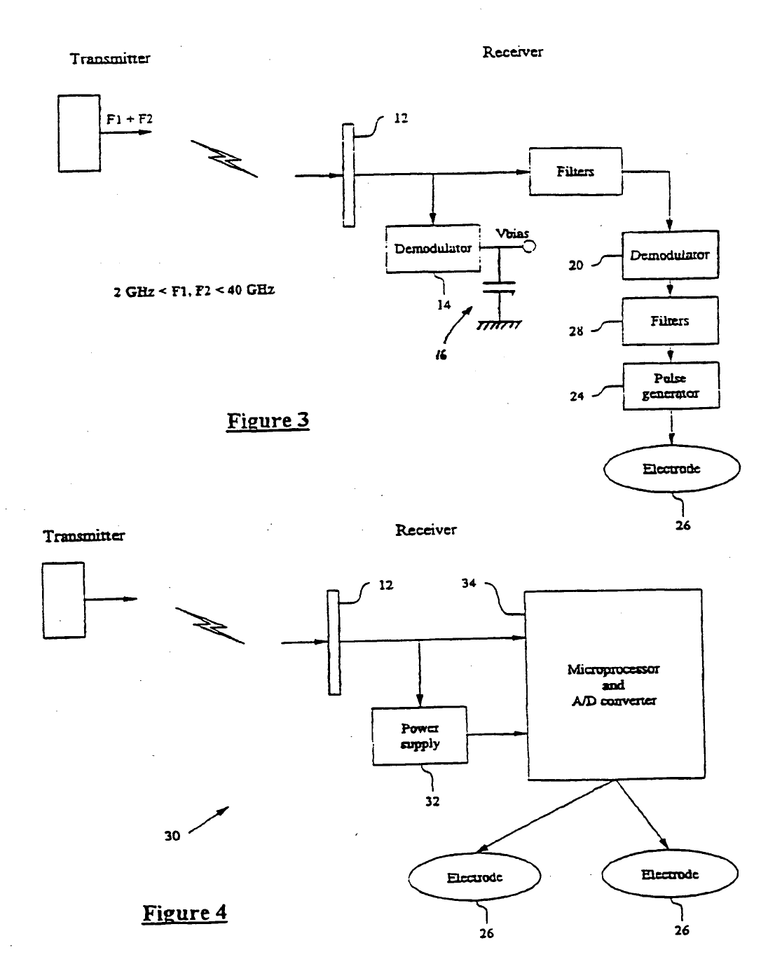

In FIG. 3, the appropriate activating device is addressed by a choice of modulating tones which is decoded by means of band pass filters 28. In this case, the duration of the tone can be used to determine the width of the pulse to be output by the pulse generator 24. The pulse then appears at the electrode plate 26 and drives a current stimulus through the tissues it is embedded in. Once again, the electrode plate may be physically next to the remainder of the activator device or may be a short distance away and coupled thereto, for example, by an insulated multistrand SS wire.

FIG. 4 illustrates in block diagram form a digital implementation of the receiver/activator 30, in which the functions of the signal filtering, demodulation, address decoding and pulse generation are all performed by a single integrated microprocessor and A/D converter circuit 34. The power for the circuit 34 is provided by the power supply circuit 32, which operates in the same manner as described hereinabove, deriving usable electrical current from the electromagnetic radiation received at the receiver antenna 12. No battery is required to provide power in this circuit. The functions of the microprocessor and A/D converter circuit are controlled by, for example, micro-coded computer program instructions in a known way. The stimulations pulses to the electrodes 26 are driven directly from the integrated circuit, and this diagram also illustrates the possibility of driving: more than one electrode from a single receiver.

Features of the device described herein include the simple construction which makes it robust and immune to the traumatic environment existing inside the body. There are no coils in the device since inductive coupling is avoided. There are no chemical reactions which is a problem in devices which have charge storage bimetallic capacitors. Lengthy wires are not required, which makes the surgical implantation procedures very simple. The device characteristics do not change if there is tissue growth, and a controllable pulse duration and stimulating current is provided for. This is useful in case where the muscle characteristics were to change whether over a long duration of time (eg through aging) or over a short duration (such as through muscle fatigue).

Because the system of the present invention does not require direct wired connections from the primary controller, numerous antenna based devices (eg stimulator devices) can be implanted without the difficulties associated with the wires bypassing joints in the subject.

For example, it is estimated that a minimum of perhaps 50 separate artificial stimulators would be required to fully restore a walking function in a subject with disabled motor functions to the legs, and wires to that many stimulator sites would be very problematic.

The present invention provides a system which can, however, easily accommodate that number of receiver/activators, with each individually addressable or addressable in selected groups. For example, with addressing of the receivers by respective digital codes, an eight bit code would enable selective activation of 256 devices and/or groups of devices.

In conjunction with FES stimulation, one further preferred aspect of the present invention also envisages a system which comprises an EMG recorder, an intelligent signal processor and an artificial stimulation controller. The purpose of this overall system is to be able to control the muscle stimulation pattern in order to provide near natural muscle contraction for subjects with neuromotor control disorder. As such this embodiment incorporates the following features:
(a) EMG measurement from the muscles under stimulation to provide feedback for controlling the artificial stimulation;
(b) processing the EMG measurements using neural network processing to extract information relating to muscle fatigue and force of muscle contraction.
(c) the ability to control the muscle stimulation based on the muscle fatigue status and the net force of contraction being produced by the muscle.

The above features can be implemented in the following manner:
(a) Neural Networks and Time frequency atoms have been used in past to analyse EMG. (Englehart K et al, “Classification of Myoelectric Signal Burst Patterns Using A Dynamic Neural Network”, IEEE 1995; Hiraiwa A et al, Shimohara K and Tokunga Y, “EMG Pattern Analysis and Classification by Neural Network” IEEE 1989, which is incorporated herein by reference; Jang G C, Cheng F H Y, Lai J S and Kuo T S “Using Time Frequency Analysis Technique in the Classification of Surface Emg Signals”, IEEE 1994, which is incorporated herein by reference). The present system utilises similar techniques to analyse EMG of the FES stimulated muscles for the purpose of having a closed loop FES system.
(b) During a training phase which is performed under supervision, a fixed stimulation pattern is applied to different electrodes in the same muscle. EMG recordings are memorised by the neural network against the muscle contraction pattern. The system learns the correlation of the EMG signal, force and fatigue. Fatigue is also taught to a parallel system with the help of the spectrum of the signal.
(c) Thereafter, the system stimulates the same muscle with the help of different pulse shapes and amplitudes and records the force of contraction. The system is self learning and this can continue even when the stimulating device is implanted. The system incorporates nested neural networks. The network learns the correlation between time, wave shape and strength of contraction.
(d) The trained system receives the EMG signal from the muscles being stimulated. The system works in a closed loop and with the help of training, it correlates time EMG wave shape and spectrum with force of contraction and fatigue. The system then changes the pulse shape and rate of muscle stimulation in order to achieve a constant muscle contraction. The system is thus able to predict and compensate for the muscle fatigue. By suitably selecting a various different sets of electrodes in the same muscle, motor recruitment can therefore be altered and muscle fatigue prevented or reduced.

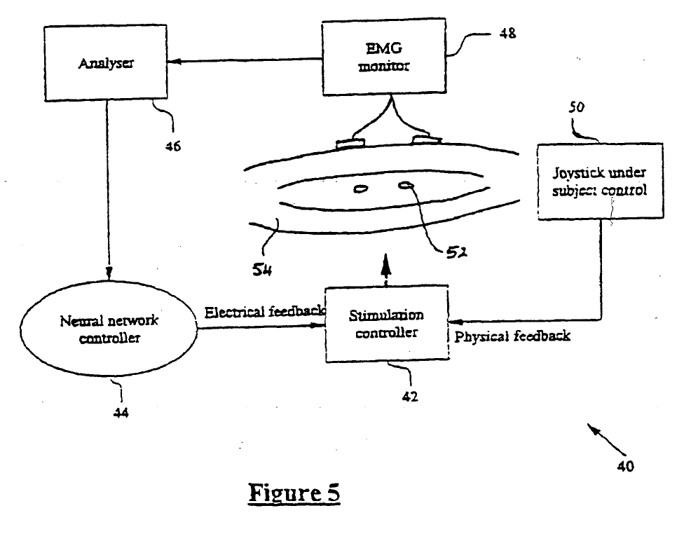

An example of an implementation of such a system 40 is illustrated in FIG. 5. A stimulation controller 42 is used to artificially stimulate the subject's muscle 54 by way of FES electrodes 52 in order to achieve muscle contraction in the subject. EMG sensors 48 measure EMG feedback signals from the muscle, which are passed to an analyser circuit 46 and thence to a neural network processor 44. The neural network processor 44 provides electrical feedback to the stimulation controller 42 according to discerned muscle fatigue, etc. A joystick 50 or the like, under control of the subject, can provide physical feedback signals indicative of, for example, muscle contraction. The above described system thus enables a technique for processing surface EMG using intelligent signal processing techniques incorporating Neural Networks. The technique extracts information related to the status of muscle fatigue and force of the stimulated muscle. The system can therefore provide information related to change in motor recruitment and stimulation in order to maintain constant force of contraction and prevent fatigue. It can also analyse the need by the subject to increase or decrease the force of contraction of any muscle.

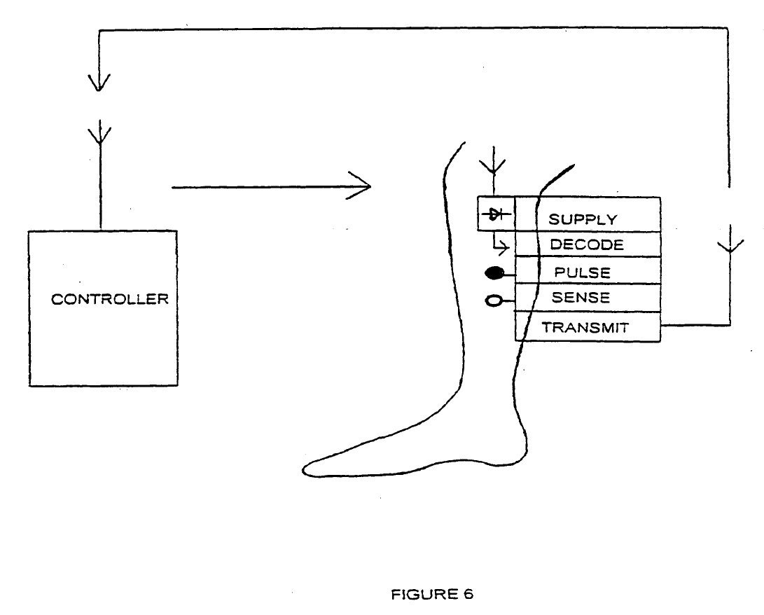

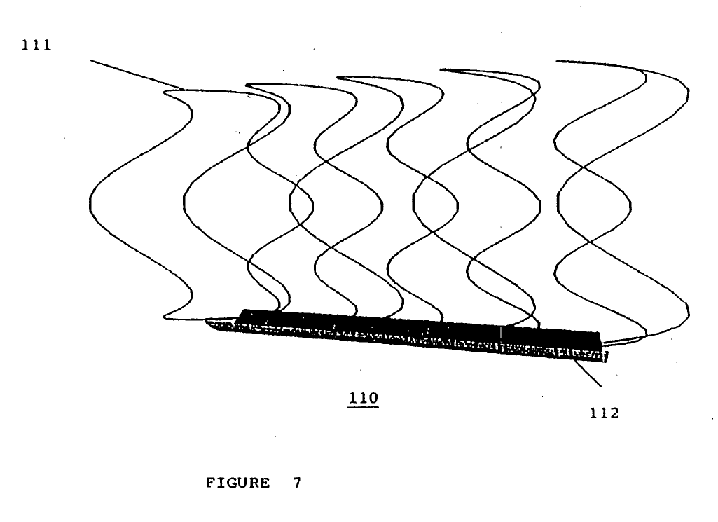

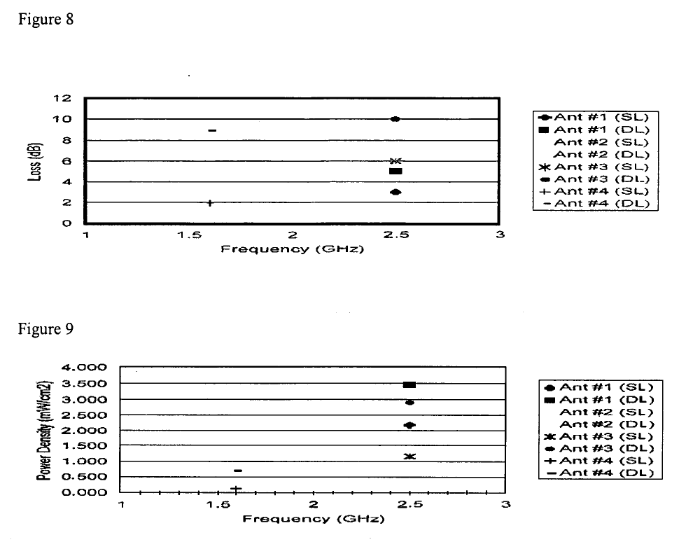

With reference to FIG. 6, the primary controller emits a signal to an implanted device in a leg. The implanted device takes the transmitted signal, decodes it, and sends out pulses for muscle stimulation as specified by the signal regulating commencement time, pulse width, pulse frequency and number of pulses. As shown the device also comprises a sensor to measure characteristics such as EMG, pH, and muscle dimensions. It then transmits data to the primary controller. In this way, the system provides a remotely powered device that can be instructed to stimulate muscles and also monitor the state of the muscles.

The medical appliance 110 in FIG. 7 has the basic elements of a known stent, that is, a spring 111 which is attached to a support 112. In this case, the support 112 is a structure capable of incorporating the elements of a monitoring device.

Due to the function of the spring 111 as an aerial for the monitoring device which is incorporated into the support 112, the amplitude of the sinusoidal pleats is kept small enough so that there is not a great deal of overlap between the loops of the spring in the stent. This prevents overlap of the electromagnetic fields generated by these individual loops.

The support 112 may therefore be at least substantially encapsulated in a biocompatible material, such as a suitable epoxy or the like. The sensors of the monitoring device may be constructed from a suitable biocompatible conductive material, such as titanium.

The medical appliance 110 in FIG. 7 is shown in its expanded form. When the medical appliance 110 is being inserted into place, the spring 111 will be in a collapsed form (not shown) to allow for easier insertion.

### Example 9

In this example, the ability to transmit power using far field energy from a primary controller to an implanted device having an antenna with a 1 cm² surface area when embedded into a leg of pork was investigated.

Methodology:

An implantable device (150) was prepared comprising a patch antenna attached to a device which measures the power received and then accepts commands to pace an attached heart device and sends a message back with the detected heart rate. The patch antenna was purchased from Spectrum Control Inc (part number PA18-1580-010SA) having dimensions of 18 mm×18 mm×4 mm. The implantable device (150) did not have a battery so its sole source of power was transmitted electromagnetic radiation from the primary controller (160). The implantable device (150) converted the electromagnetic radiation into DC energy and stored it in a super-capacitor which could then provide up to 20 minutes of continuous operation after the primary controller (160) stopped transmitting. The super-capacitor was not charged prior to the experiment so all its energy was received from the primary controller.

The implantable device (150) received instructions from the primary controller (160). In this example, the implantable device (150) delivered heart pacemaker type stimuli through bipolar electrodes. The implantable device (150) also provided data back to the primary controller (160) through a back channel receiver (165) by remote signals. The back channel receiver (165) was placed at a distance of 10 m from the implantable device (150).

The implantable device was implanted in a raw leg of pork (170) under the skin and fat at a depth of about 1 cm.

The primary controller (160) transmitted energy at a frequency in the range of 1.5 to 1.6 GHz and was positioned at a distance of at least 4 meters from the leg of pork (170) containing the implantable device (150). The output power of the power amplifier (180) was measured in Watts.

A laptop (190) was attached to the primary controller (160) to send commands to the implantable device (150) by modulating the same electromagnetic radiation used for powering the implantable device (150).

The back channel receiver (165) was a wireless channel used to transmit measurements of the actual stimulation rate across the bipolar electrode as well as measurements of the energy level of the super-capacitor.

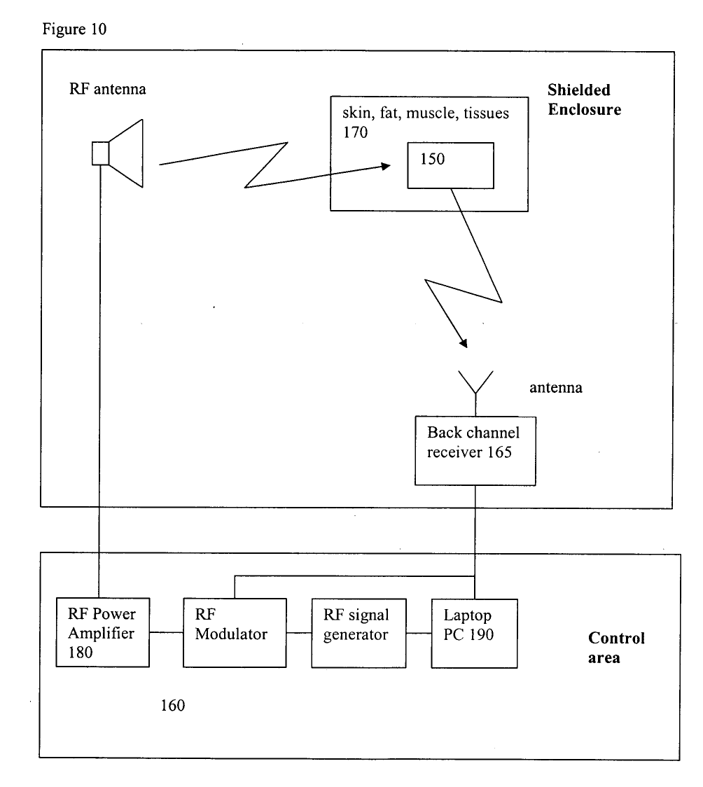

FIG. 10 illustrates in block diagram form the set up used during the experiment. The experiment was conducted inside a shielded enclosure to prevent any interference to/from external factors.

After receiving electromagnetic energy from the primary controller (160) for about 4 minutes, the super-capacitor in the implantable device (150) was sufficiently charged to commence operation of the implantable device (150). Whilst in operation, the implanted device (150) was tested for proper operation by sending interrogation commands which then triggers a response from the implanted device (150). Depending on the specific command, the response of the implantable device (150) consisted of the voltage level of the super-capacitor or the stimulation rate at the electrodes. The artificial stimulation rate could be chosen between two rates or be stopped altogether.

All responses of the implantable device (150) were tested against various electromagnetic power levels and distances from the antennae. Field strength levels next to the implantable device (150) were also recorded.

Results

The implantable device (150) was demonstrated to be powered by electromagnetic waves and achieved full functionality at field strengths as low as 30 Vpk/m. This field strength corresponds to a power density of 0.12 mW/cm², which is 8 times lower than the safety level for general public as per OET Bulletin 65 (FCC—Office of Engineering & Technology), which is incorporated herein by reference. Power densities required for operating a state of the art device are expected to be much lower. Consequently, the necessary transmission power would be reduced and/or the distance to the implantable device would be increased significantly.

The implantable device measured voltages in the range of 1.5 to 2.5 volts. The system had a maximum capacity of 2.5 volts.

An ECG machine independently confirmed the heart rate to be 70 Bpm when resting and 100 Bpm when the pacing device was instructed to pace a higher rate.

This experiment proves that a system according to the invention can successfully transmit power through space (air) to penetrate through skin and fat and continue to penetrate into the tissue.

### Example 10

This example investigated the ability to transmit power using far field energy from a primary controller to an implanted device having an antenna which was the spring of a stent.

The methodology of example 9 was followed.

The frequencies used were in the range from 1.4 to 1.8 GHz. The stent was made of wire of varying lengths thereby creating loops of 3-5 mm. Such stents are very small and capable of being fully implanted in small blood vessels such as the heart coronary arteries or veins. These vessels have a much smaller diameter than the aorta (cm) where prior art devices have been used. The implanted device was completely wireless and was not directly connected to a battery or larger device. It was powered solely by remote via electromagnetic radiation which was converted to DC by a diode.

This power could be used to drive microelectronics or microchips attached as sensors of cardiac activity (rate, regularity, blood pH) or as a stimulating device to pace/control heart rate.

### Comparative Example 11

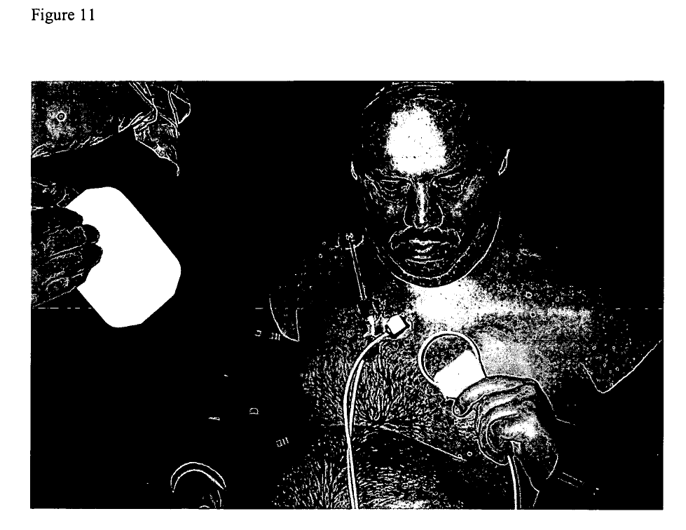

FIG. 11 is a photo of a patient using a prior art implanted device. It is clear from this photo that the antenna of the external controller must be aligned over the implanted device and be within very close proximity to the skin of the patient. Such a device is not suitable for use with more than one implanted device or devices implanted deep into the body.

It is to be understood by those skilled in the technology that many variations or modifications in details of design or construction may be made without departing from the essence of the present invention. Therefore, the invention should be understood to comprise all such variations and modifications within its scope. Further, whilst the applications of the invention have been described in relation to the human body, they are equally applicable to other living bodies such as animals.

All patents, patent applications and publications cited herein are hereby incorporated by reference regardless of whether incorporated above.

It is specifically understood that any numerical range recited herein includes all values from the lower value to the upper value, i.e., all possible combinations of numerical values between the lowest value and the highest value enumerated are to be considered to be expressly stated in this application. For example, if a distance range is stated as up to 12 m, it is intended that values such as 50 cm, 100 cm, 1 m, 5 m, 7 m and 10 m, etc., are expressly enumerated in this specification. These are only examples of what is specifically intended.

The word ‘comprising’ as used in this description and in the claims does not limit the invention claimed to exclude any variants or additions.

1. A system for transmission of power between a first location external of a living body and a second position internal of the living body which comprises:
(a) a primary controller comprising a power source and a transmitter, locatable at the first location; and
(b) an antenna based device locatable at the second position to receive an output from the transmitter, said antenna based device including a capacitor,
wherein the power source is adapted to emit high frequency electromagnetic radiation between 0.5 to 5 GHz; and
wherein, in use, the antenna based device converts the transmitted power to electrical energy which is used to operate the antenna based device,
wherein the primary controller and the antenna based device do not communicate by inductive coupling; and
wherein the primary controller is capable of transmitting power to the antenna based device from at least about 50 cm.

2. The system according to claim 1, wherein there is transmission of information as well as power, and wherein the primary controller further comprises a receiver to receive data from the implanted device.

3. The system according to claim 1 wherein the power source in the primary controller is adapted to emit high frequency radiation between 0.8 to 3.5 GHz.

4. The system according to claim 1 wherein the power source in the primary controller is adapted to emit high frequency radiation between 0.8 to 2.5 GHz.

5. The system according to claim 1 wherein the power source in the primary controller is adapted to emit high frequency radiation between 1.5 to 3.5 GHz.

6. The system according to claim 1 wherein the antenna based device comprises an antenna which has a planar omnidirectional format and is integrated into the construction of the antenna based device.

7. The system according to claim 1 wherein the antenna based device comprises an antenna which has a format selected from the group consisting of a simple dipole, a loop with or without crenellations, or a microstrip antenna including slot and patch formats.

8. The system according to claim 7 wherein the antenna is a patch antenna having a surface area of 1 cm².

9. The system according to claim 7 wherein the antenna is a loop having a diameter of 3 to 5 mm.

10. The system according to claim 1 wherein the primary controller further comprises other devices.

11. The system according to claim 1 wherein the antenna based device further comprises means to monitor predetermined conditions adjacent the antenna based device and to emit signals representative of one or more of these conditions to be received by the primary controller.

12. The system according to claim 1 wherein the antenna based device further comprises means to generate pulses of current.

13. The system according to claim 1 wherein, in use, the first location is separated from the second position by a distance of up to 12 metres.

14. The system according to claim 13 wherein the distance is in the range of about 4 metres.

15. The system according to claim 1 wherein the antenna based device is a medical appliance.

16. The system according to claim 15 wherein the antenna based device is a stent.

17. The system according to claim 16 wherein the stent is spring-based and the spring of the stent acts as an antenna, and wherein the stent incorporates a monitoring device.

18. The system according to claim 17 wherein the monitoring device is located in the support of the stent.

19. The system according to claim 17 wherein the monitoring device further comprises means to monitor predetermined conditions in the vicinity of the stent.

20. The system according to claim 17 wherein the monitoring device works in conjunction with the primary controller.

21. The system according to claim 17 wherein the monitoring device further comprises means to emit signals representative of one or more of these conditions to be received by the primary controller.

22. The system according to claim 17 further comprising an intermediate implant which relays power and/or information from the primary controller to the stent.

23. The system according to claim 15 wherein the antenna based device is a wireless electrode and the primary controller is a pacemaker.

24. The system according to claim 15 wherein the antenna based device is an electrode for implanting in a patient's brain.

25. The system according to claim 15 wherein the antenna based device is adapted to be implanted in, or adjacent to, a patient's eye to provide artificial sight.

26. A method for transmitting power between a first location external of a living body at which a primary controller is located, wherein the primary controller comprises a power source and a transmitter and a second position internal of the living body at which the antenna based device is located, said antenna based device comprising a capacitor, the method comprising the steps of:
(a) generating high frequency electromagnetic radiation between 0.5 to 5 GHz from the power source and emitting that radiation from the transmitter of the primary controller,
(b) receiving the radiation at the antenna based device, and
(c) powering the antenna based device with the radiation;
wherein the primary controller and antenna-based device do not communicate by inductive coupling; and
wherein the primary controller is capable of transmitting power to the antenna based device from at least about 50 cm.

27. The method according to claim 26 wherein the high frequency radiation in step (a) is 0.8 to 2.5 GHz.

28. The method according to claim 26 wherein the method comprises further steps of:
(d) causing the antenna based device to generate and emit pulses of current; and/or
(e) monitoring predetermined conditions adjacent to the antenna based device and emitting signals representative of one or more of these conditions to be received by the primary controller.

29. The system according to claim 15 wherein the medical appliance is a stimulating device for providing artificial stimulation to a muscle.

30. The system according to claim 29 further comprising an electromyogram sensor for measuring electromyogram signals from the muscle during stimulation and a neural network processor coupled to receive the measured electromyogram signals to extract information regarding force of contraction and fatigue of the muscle, wherein the primary controller is coupled to an output of the neural network processor to control said artificial electrical stimulation based on said extracted information.

31. A method for implementing the system according to claim 29 comprising the steps of:
(a) performing a training phase under supervision wherein a fixed stimulation pattern is applied to different electrodes in the same muscle; electromyogram recordings are memorised by the neural network against the muscle contraction pattern; and the system learns the correlation of the electromyogram signal, force and fatigue;
(b) thereafter, recording the force of contraction when the same muscle is stimulated with different pulse shapes and amplitudes;
(c) correlating the time electromyogram wave shape and spectrum of electromyogram signals received from the muscle being stimulated with force of contraction and fatigue; and
(d) changing the pulse shape and rate of stimulation in order to achieve a constant muscle contraction.
